(19) United States
(12) Patent Application Publication
(10) Pub. No.: US 2023/0347272 A1
(43) Pub. Date: Nov. 2, 2023

(54) CABIN AIR FILTERS FOR VEHICLES

(71) Applicant: Donald Luttrell, Basehor, KS (US)

(72) Inventor: Donald Luttrell, Basehor, KS (US)

(21) Appl. No.: 17/981,211

(22) Filed: Nov. 4, 2022

Related U.S. Application Data
(60) Provisional application No. 63/335,305, filed on Apr. 27, 2022.

(51) Int. Cl. B01D 46/52; B01D 46/00; B01D 39/18; A61L 9/04; B60H 3/06; A61L 9/12

(52) U.S. Cl. CPC B01D 46/0038; A61L 9/042; A61L 9/12; B01D 39/18; B01D 46/0002; B01D 46/521; B60H 3/0608; A61L 2209/14; B01D 2239/045; B01D 2279/50

(57) ABSTRACT

A cabin air filter for use in cabins of vehicles for air filtration and conditioning of indoor environment in the cabin. The cabin air filter includes a filter media encased in a frame and a fragrance formulation deposited on the filter media.

### CROSS-REFERENCE TO RELATED APPLICATIONS

This application claims priority from a U.S. Provisional Pat. Appl. No. 63/335,305 filed on Apr. 27, 2022, which is incorporated herein by reference in its entirety.

### FIELD OF INVENTION

The present invention relates to a cabin air filter for a vehicle, and more particularly, the present invention relates to a cabin air filter with fragrance.

### BACKGROUND

Cabin air filters are used in vehicles to clean indoor air in the cabin of the vehicle from dust, pollens, and the like contaminants in the indoor air. Besides cabin air filters, owners of the vehicle also use perfumes and the like in their vehicles for fragrance and masking bad odour. The fragrance units are mounted such that air from cabin air filters can pass through the fragrance units. However, finding an optimum position for fragrance units and mounting the fragrance units at the optimum locations can be complex and often not possible.

A need is therefore appreciated for a device that overcomes the aforesaid drawbacks and limitations with fragrance units for use in vehicles.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a cabin air filter incorporated with fragrance for vehicles.

It is another object of the present invention that separate air fragrance units are not needed.

It is still another object of the present invention that the overall cost of the cabin air filter and fragrance unit can be reduced.

It is a further object of the present invention that the wastage is reduced.

It is yet another object of the present invention that the cabin air filter can be retrofitted.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention since the scope of the invention will be best defined by the allowed claims of any resulting patent.

In the following description, for purposes of explanation, specific details may be set forth in order to provide a thorough understanding of the subject innovation. It may be evident, however, that the claimed subject matter may be practiced without these specific details. In other instances, well-known structures and apparatus are shown in block diagram form in order to facilitate describing the subject innovation.

Disclosed is a cabin air filter incorporated with fragrance formulation. The cabin air filter can be made from paper and like material known to a skilled person for use in air filters, and any such material is within the scope of the present invention. For example, the filter material used can be paper, cloth, fiberglass, and the like. The filter media made from paper and cloth can be pleated to increase the surface area for the filtration of air. The filter media can be encased in a frame, such as frames made from plastic, wood, aluminium, and the like can be used. The moulded plastic frames can be economical and can be easily scaled up and thus is preferable.

The cabin air filter can be ingrained with suitable fragrances. The fragrance can be incorporated in a controlled release form, such that the fragrance is released at a predetermined rate for months. For example, the life of the filter media and the fragrance incorporated in the filter media can be matched. Suitable forms of fragrance can be beads through which the fragrance can be released slowly by the air in a controlled manner. Suitable polymers can be used for the controlled release of the fragrance. The filer media, such as paper can be embedded with the controlled release formulation of fragrance. The air passing through the disclosed cabin air filter can be filtered by the filter media and then carry the fragrance to the interior of the cabin. The controlled release formulation of the fragrance can be incorporated at the side of the filter media through which filtered air exits.

The disclosed filter media can be manufactured in a range of fragrances, such as but not limited to flower extracts, cinnamon lemon, vanilla, orange, leather, pine, cologne, citrus, peach, cherry, apple, watermelon, coconut, and blueberry. The type of fragrance of the cabin air filter can be printed on the label of the cabin air filter. The disclosed cabin air filters can be made in a range of sizes and shapes for different air filtration setups in different vehicles. The disclosed cabin air filter can be incorporated into new air filtration units as well as retrofitted into old air filtration units as a replacement air filter. The pore size of the filtration media can be varied for filtering particles of different sizes without departing from the scope of the present invention.

It is to be noted that preferred embodiments have been described for cabin air filters for use in vehicles, however, the disclosed cabin air filter can be adapted for use in other air filtration setups, such as room air purifiers for resident and commercial uses, HVAC units, and the like.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A cabin air filter comprising:
a filter media encased in a frame; and
a fragrance formulation deposited on the filter media.

2. The cabin air filter according to claim 1, wherein the filter media is made from pleated filter paper.

3. The cabin air filter according to claim 1, wherein the fragrance formulation is selected from a group consisting of flower extracts, cinnamon, lemon, vanilla, orange, leather, pine, cologne, citrus, peach, cherry, apple, watermelon, coconut, and blueberry.

4. The cabin air filter according to claim 1, wherein the fragrance formulation is controlled release.

5. A method for releasing fragrance in a cabin of a vehicle, the method comprises:
providing a cabin air filter comprising:
a filter media encased in a frame, and
a fragrance formulation deposited on the filter media; and
installing the cabin air filter in a cabin’s air filtration setup of the vehicle.

6. The method according to claim 5, wherein the filter media is made from pleated filter paper.

7. The method according to claim 5, wherein the fragrance formulation is selected from a group consisting of flower extracts, cinnamon, lemon, vanilla, orange, leather, pine, cologne, citrus, peach, cherry, apple, watermelon, coconut, and blueberry.

8. The method according to claim 5, wherein the fragrance formulation is a controlled release formulation.
